(19) United States
(12) Patent Application Publication
(10) Pub. No.: US 2021/0331145 A1
(43) Pub. Date: Oct. 28, 2021

(54) HYDROGENATION PROCESS COMPRISING A CATALYST PREPARED BY ADDITION OF AN ORGANIC COMPOUND IN THE GAS PHASE

(71) Applicant: IFP Energies nouvelles, Rueil-Malmaison Cedex (FR)

(72) Inventors: Anne-Claire DUBREUIL, Rueil-Malmaison Cedex (FR); Vincent COUPARD, Rueil-Malmaison Cedex (FR); P-Louis CARRETTE, Rueil-Malmaison Cedex (FR); Florent GUILLOU, Rueil-Malmaison Cedex (FR); Bertrand GUICHARD, Rueil-Malmaison Cedex (FR)

(73) Assignee: IFP Energies nouvelles, Rueil-Malmaison Cedex (FR)

(21) Appl. No.: 17/281,067

(22) PCT Filed: Oct. 15, 2019
(86) PCT No.: PCT/EP2019/078006

§ 371 Date: Mar. 29, 2021

(30) Foreign Application Priority Data
Oct. 25, 2018 (FR) 1871300

(51) Int. Cl. B01J 23/755; B01J 21/04; B01J 21/18; B01J 37/02; B01J 37/08; C10G 45/36; C07C 5/10

(52) U.S. Cl. CPC B01J 23/755; C07C 2521/18; B01J 21/18; B01J 37/0205; B01J 37/0203; B01J 37/0236; B01J 37/084; B01J 37/088; C10G 45/36; C07C 5/10; C10G 2300/301; C10G 2300/4006; C10G 2300/4012; C10G 2300/4018; C10G 2300/70; C07C 2523/755; C07C 2521/04; B01J 21/04

(57) ABSTRACT

A process for the hydrogenation of a polyunsaturated compound contained in a hydrocarbon feedstock in the presence of a catalyst comprising a porous support and an active phase comprising a group VIII metal, said catalyst being prepared according to the following steps:
a) an organic compound containing oxygen and/or nitrogen, but not comprising sulfur, is added to the porous support;
b) said porous support is brought into contact with a solution containing a salt of a precursor of the active phase;
c) the porous support obtained at the end of step b) is dried;
   characterized in that step a) is carried out before or after steps b) and c) and is carried out by bringing together said porous support and said organic compound under conditions of temperature, pressure and duration such that a fraction of said organic compound is transferred in the gaseous state to the porous support.

### TECHNICAL FIELD

The subject of the invention is a process for the selective hydrogenation of polyunsaturated compounds in a hydrocarbon-based feedstock, in particular in the C2-C5 steam cracking fractions and steam cracking gasolines, or a process for the hydrogenation of at least one aromatic or polyaromatic compound contained in a hydrocarbon-based feedstock allowing the transformation of aromatic compounds from petroleum or petrochemical fractions by conversion of the aromatic nuclei into naphthenic nuclei. The process for the selective hydrogenation or the hydrogenation of aromatics is carried out in the presence of a catalyst prepared according to a particular procedure.

### PRIOR ART

Catalysts for the selective hydrogenation of polyunsaturated compounds or for the hydrogenation of aromatic compounds are generally based on metals from group VIII of the Periodic Table of Elements, such as nickel. The metal is in the form of nanometric metal particles deposited on a support which may be a refractory oxide. The content of group VIII metal, the optional presence of a second metal element, the size of the metal particles and the distribution of the active phase in the support and also the nature and the pore distribution of the support are parameters which may have an influence on the performance of the catalysts.

The rate of the hydrogenation reaction is governed by several criteria, such as the diffusion of the reactants at the surface of the catalyst (external diffusional limitations), the diffusion of the reactants in the porosity of the support toward the active sites (internal diffusional limitations) and the intrinsic properties of the active phase, such as the size of the metal particles and the distribution of the active phase within the support.

As regards the size of the metal particles, it is generally accepted that the catalyst becomes more active as the size of the metal particles decreases. Furthermore, it is important to obtain a particle size distribution which is centered on the optimum value and also a narrow distribution around this value.

The most conventional route for the preparation of these catalysts is the impregnation of the support with an aqueous solution of a nickel precursor, generally followed by a drying and a calcination. Before they are used in hydrogenation reactions, these catalysts are generally reduced in order to obtain the active phase, which is in the metallic form (that is to say, in the zero valency state). Catalysts based on nickel on alumina prepared by just one impregnation step generally make it possible to achieve nickel contents of between 12% and 15% by weight of nickel approximately, with respect to the total weight of the catalyst, depending on the pore volume of the alumina used. If it is desired to prepare catalysts having a higher nickel content, several successive impregnations are often necessary in order to obtain the desired nickel content, followed by at least one drying step and then optionally by a calcination step between each impregnation.

Furthermore, for the purpose of obtaining better catalytic performance properties, especially better selectivity and/or activity, it is known in the prior art to use additives of organic compound type for the preparation of metal catalysts, especially for catalysts which were prepared by impregnation optionally followed by a maturation step and followed by a drying step. Many documents describe the use of various ranges of organic compounds, such as nitrogen-containing organic compounds and/or oxygen-containing organic compounds. For example, application FR2984761 discloses a process for the preparation of a selective hydrogenation catalyst comprising a support and an active phase comprising a group VIII metal, said catalyst being prepared by a process comprising a step of impregnation with a solution containing a precursor of the group VIII metal and an organic additive, more particularly an organic compound having from one to three carboxylic acid functions, a step of drying the impregnated support, and a step of calcining the dried support in order to obtain the catalyst.

The processes for preparing additivated catalysts typically implement an impregnation step wherein the organic compound is introduced, optionally in solution in a solvent, so as to fill all the porosity of the support, whether or not it is impregnated with metallic precursors, in order to obtain a homogeneous distribution. This inevitably results in using large amounts of organic compound or in diluting the organic compound in a solvent. After impregnation, a drying step is then necessary to eliminate the excess compound or the solvent and thus free the porosity needed for the use of the catalyst. Added to the additional cost linked to the excess organic compound or to the use of a solvent is the cost of an additional, energy-consuming separate preparation step of drying. During the drying step, the evaporation of the solvent may also be accompanied by a partial loss of the organic compound by vaporization and therefore by a loss of catalytic activity.

The Applicant has surprisingly discovered that a catalyst comprising an active phase based on at least one group VIII metal, preferably nickel, supported on an oxide matrix, prepared from a preparation process comprising at least one step of adding an organic compound to the porous support by gas-phase impregnation makes it possible to obtain performance levels in terms of activity with respect to selective hydrogenation of polyunsaturated compounds or to hydrogenation of aromatic compounds that are at least as good as, or even better than, the processes known from the prior art. Without wishing to be bound by any theory, it seems that the gaseous addition of the organic additive during the preparation of the catalyst makes it possible to obtain hydrogenation performance levels in terms of activity that are at least as good as, or even better than, known catalysts, the preparation process of which comprises a step of adding one and the same organic additive by the liquid route (for example by dry impregnation) even though the size of the particles of active phase obtained on the catalyst (measured in their oxide forms) is equivalent.

### Subjects of the Invention

A subject of the present invention is a process for the hydrogenation of at least one polyunsaturated compound containing at least 2 carbon atoms per molecule, such as diolefins and/or acetylenics and/or aromatic or polyaromatic compounds, contained in a hydrocarbon feedstock having a final boiling point of less than or equal to 650° C., which process being carried out at a temperature of between 0 and 350° C., at a pressure of between 0.1 and 20 MPa, at a hydrogen/(compound to be hydrogenated) molar ratio between 0.1 and 1000 and at an hourly space velocity HSV of between 0.05 and 40 000 h⁻¹ in the presence of a catalyst comprising a porous support and an active phase comprising at least one group VIII metal, said active phase not comprising a group VIB metal, said catalyst being prepared according to at least the following steps:
a) at least one organic compound containing oxygen and/or nitrogen, but not comprising sulfur, is added to the porous support;
b) a step of bringing said porous support into contact with at least one solution containing at least one salt of a precursor of the phase comprising at least one group VIII metal is carried out;
c) the porous support obtained at the end of step b) is dried;
   characterized in that step a) is carried out before or after steps b) and c) and is carried out by bringing together said porous support and said organic compound under conditions of temperature, pressure and duration such that a fraction of said organic compound is transferred in the gaseous state to the porous support.

In one embodiment according to the invention, step a) is carried out by simultaneously bringing together said porous support and said organic compound in the liquid state and without physical contact, at a temperature below the boiling point of said organic compound and under conditions of pressure and duration such that a fraction of said organic compound is transferred in the gaseous state to the porous support.

Advantageously, step a) is carried out by means of a unit for adding said organic compound comprising a first compartment and a second compartment that are in communication so as to allow the passage of a gaseous fluid between the compartments, the first compartment containing the porous support and the second compartment containing the organic compound in the liquid state.

Advantageously, the unit comprises a chamber including the first and second compartments, the two compartments being in gaseous communication.

Advantageously, the unit comprises two chambers that respectively form the first and the second compartments, the two chambers being in gaseous communication.

Advantageously, step a) is carried out in the presence of a stream of a carrier gas circulating from the second compartment into the first compartment.

In a second embodiment according to the invention, step a) is carried out by bringing together said porous support with a porous solid comprising said organic compound under conditions of temperature, pressure and duration such that a fraction of said organic compound is transferred gaseously from said porous solid to said porous support.

Preferably, step a) is carried out by bringing together, without physical contact, said porous support with a porous solid comprising said organic compound.

Preferably, in step a), the porous support and the porous solid comprising said organic compound are of different porosity and/or of different chemical nature.

Preferably, at the end of step a), the porous solid containing the organic compound is separated from said porous support and is returned to step a).

Advantageously, said organic compound is chosen from compounds comprising one or more chemical functions chosen from a carboxylic acid, alcohol, ester, aldehyde, ketone, ether, carbonate, amine, azo, nitrile, imine, amide, carbamate, carbamide, amino acid, ether, dilactone or carboxyanhydride function.

In one embodiment according to the invention, the process is a process for the hydrogenation of at least one aromatic or polyaromatic compound contained in a hydrocarbon feedstock having a final boiling point of less than or equal to 650° C., said process being carried out in the gas phase or in the liquid phase, at a temperature of between 30 and 350° C., at a pressure of between 0.1 and 20 MPa, at a hydrogen/(aromatic compounds to be hydrogenated) molar ratio between 0.1 and 10 and at an hourly space velocity HSV of between 0.05 and 50 h⁻¹.

In one embodiment according to the invention, the process is a process for the selective hydrogenation of polyunsaturated compounds contained in a hydrocarbon feedstock having a final boiling point of less than or equal to 300° C., which process being carried out at a temperature of between 0 and 300° C., at a pressure of between 0.1 and 10 MPa, at a hydrogen/(polyunsaturated compounds to be hydrogenated) molar ratio of between 0.1 and 10 and at an hourly space velocity of between 0.1 and 200 h⁻¹ when the process is carried out in the liquid phase, or at a hydrogen/(polyunsaturated compounds to be hydrogenated) molar ratio of between 0.5 and 1000 and at an hourly space velocity of between 100 and 40 000 h⁻¹ when the process is carried out in the gas phase.

### BRIEF DESCRIPTION OF THE FIGURES

FIG. 1 schematically illustrates an embodiment of step a) of the process for preparing the catalyst used in the context of the hydrogenation process according to the invention.

### DETAILED DESCRIPTION

### Definitions

“Macropores” is understood to mean pores the opening of which is greater than 50 nm.

“Mesopores” is understood to mean pores the opening of which is between 2 nm and 50 nm, limits included.

“Micropores” is understood to mean pores the opening of which is less than 2 nm.

The term “total pore volume” of the catalyst or of the support used for the preparation of the catalyst according to the invention is intended to mean the volume measured by intrusion with a mercury porosimeter according to Standard ASTM D4284-83 at a maximum pressure of 4000 bar (400 MPa), using a surface tension of 484 dyne/cm and a contact angle of 140°. The wetting angle was taken equal to 140° following the recommendations of the work “Techniques de l'ingénieur, traité analyse et caractérisation” [Techniques of the Engineer, Analysis and Characterization Treatise], pages 1050-1055, written by Jean Charpin and Bernard Rasneur.

In order to obtain better accuracy, the value of the total pore volume corresponds to the value of the total pore volume measured by intrusion with a mercury porosimeter measured on the sample minus the value of the total pore volume measured by intrusion with a mercury porosimeter measured on the same sample for a pressure corresponding to 30 psi (approximately 0.2 MPa).

The term “specific surface area” of the catalyst or of the support used for the preparation of the catalyst according to the invention is understood to mean the BET specific surface area determined by nitrogen adsorption in accordance with Standard ASTM D 3663-78 drawn up from the Brunauer-Emmett-Teller method described in the journal “The Journal of the American Chemical Society”, 60, 309 (1938).

The term “size of the nickel nanoparticles” is understood to mean the mean diameter of the nickel crystallites measured in their oxide forms. The mean diameter of the nickel crystallites in oxide form is determined by X-ray diffraction, from the width of the diffraction line located at the angle 2θ=43° (i.e. along the crystallographic direction [200]) using the Scherrer equation. This method, used in X-ray diffraction on polycrystalline samples or powders, which links the full width at half maximum of the diffraction peaks to the size of the particles, is described in detail in the reference: Appl. Cryst. (1978), 11, 102-113, “Scherrer after sixty years: A survey and some new results in the determination of crystallite size”, J. I. Langford and A. J. C. Wilson.

In the remainder of the text, the groups of chemical elements are given according to the CAS classification (CRC Handbook of Chemistry and Physics, published by CRC Press, editor D. R. Lide, 81st edition, 2000-2001). For example, group VIII according to the CAS classification corresponds to the metals of columns 8, 9 and 10 according to the new IUPAC classification.

Description of the Catalyst Preparation Process

In general, the process for preparing the catalyst used in the context of the hydrogenation process according to the invention comprises at least the following steps:
a) at least one organic compound containing oxygen and/or nitrogen, but not comprising sulfur, is added to a porous support;
b) a step of bringing said porous support into contact with at least one solution containing at least one salt of a precursor of the active phase comprising at least one group VIII metal is carried out;
c) the porous support obtained at the end of step b) is dried;
   characterized in that step a) is carried out:
   before or after steps b) and c); and
   by bringing together said porous support and said organic compound under conditions of temperature, pressure and duration such that a fraction of said organic compound is transferred in the gaseous state to the porous support.

Steps a) to c) of the process for preparing the catalyst used in the context of the hydrogenation process according to the invention are described in more detail below.

Step a)

Any organic compound containing oxygen and/or nitrogen but not comprising sulfur which is in the liquid state at the temperature and at the pressure that are implemented in the step of adding the organic compound to the porous support may be used in the process for preparing the catalyst.

Preferably, said organic compound is chosen from a compound comprising one or more chemical functions chosen from a carboxylic acid, alcohol, ester, aldehyde, ketone, ether, carbonate, amine, azo, nitrile, imine, amide, carbamate, carbamide, amino acid, ether, dilactone or carboxyanhydride function.

When said organic compound comprises at least one carboxylic function, said organic compound can be chosen from formic acid, ethanedioic acid (oxalic acid), propanedioic acid (malonic acid), pentanedioic acid (glutaric acid), hydroxyacetic acid (glycolic acid), 2-hydroxypropanoic acid (lactic acid), 2-hydroxypropanedioic acid (tartronic acid), 2-hydroxybutanedioic acid (malic acid), 2-hydroxypropane-1,2,3-tricarboxylic acid (citric acid), 2,3-dihydroxybutanedioic acid (tartaric acid), 2,2′-oxydiacetic acid (diglycolic acid), 2-oxopropanoic acid (pyruvic acid) and 4-oxopentanoic acid (levulinic acid).

When said organic compound comprises at least one alcohol function, said organic compound can be chosen from methanol, ethanol, phenol, ethylene glycol, propane-1,3-diol, butane-1,4-diol, pentane-1,5-diol, hexane-1,6-diol, glycerol, xylitol, mannitol, sorbitol, pyrocatechol, resorcinol, hydroquinol, diethylene glycol, triethylene glycol, polyethylene glycols having an average molar mass of less than 600 g/mol, glucose, mannose, fructose, sucrose, maltose and lactose, in any of their isomeric forms.

When said organic compound comprises at least one ester function, said organic compound can be chosen from a γ-lactone or a δ-lactone containing between 4 and 8 carbon atoms, the γ-butyrolactone, γ-valerolactone, δ-valerolactone, γ-caprolactone, δ-caprolactone, γ-heptalactone, δ-heptalactone, γ-octalactone, δ-octalactone, methyl methanoate, methyl acetate, methyl propanoate, methyl butanoate, methyl pentanoate, methyl hexanoate, methyl octanoate, methyl decanoate, methyl laurate, methyl dodecanoate, ethyl acetate, ethyl propanoate, ethyl butanoate, ethyl pentanoate, ethyl hexanoate, dimethyl oxalate, dimethyl malonate, dimethyl succinate, dimethyl glutarate, dimethyl adipate, diethyl oxalate, diethyl malonate, diethyl succinate, diethyl glutarate, diethyl adipate, dimethyl methylsuccinate, dimethyl 3-methylglutarate, methyl glycolate, ethyl glycolate, butyl glycolate, benzyl glycolate, methyl lactate, ethyl lactate, butyl lactate, tert-butyl lactate, ethyl 3-hydroxybutyrate, ethyl mandelate, dimethyl malate, diethyl malate, diisopropyl malate, dimethyl tartrate, diethyl tartrate, diisopropyl tartrate, trimethyl citrate, triethyl citrate, ethylene carbonate, propylene carbonate, trimethylene carbonate, diethyl carbonate, diphenyl carbonate, dimethyl dicarbonate, diethyl dicarbonate and di-tert-butyl dicarbonate, in any of their isomeric forms.

When the organic compound comprises at least one amine function, said organic compound can be chosen from ethylenediamine, diaminohexane, tetramethylenediamine, hexamethylenediamine, tetramethylethylenediamine, tetraethylethylenediamine, diethylenetriamine and triethylenetetramine.

When the organic compound comprises at least one amide function, said organic compound can be chosen from formamide, N-methylformamide, N,N-dimethylformamide, N-ethylformamide, N,N-diethylformamide, acetamide, N-methylacetamide, N,N-dimethylmethanamide, N,N-diethylacetamide, N,N-dimethylpropionamide, propanamide, 2-pyrrolidone, N-methyl-2-pyrrolidone, γ-lactam, caprolactam, acetylleucine, N-acetylaspartic acid, aminohippuric acid, N-acetylglutamic acid, 4-acetamidobenzoic acid, lactamide and glycolamide, urea, N-methylurea, N,N′-dimethylurea, 1,1-dimethylurea and tetramethylurea, according to any one of their isomeric forms.

When the organic compound comprises at least one amino acid function, said organic compound can be chosen from alanine, arginine, lysine, proline, serine, threonine and EDTA (ethylenediaminetetraacetic acid).

When the organic compound comprises at least one ether function, said organic compound can be chosen from the group of linear ethers consisting of diethyl ether, dipropyl ether, dibutyl ether, methyl tert-butyl ether, diisopropyl ether, di-tert-butyl ether, methoxybenzene, phenyl vinyl ether, isopropyl vinyl ether and isobutyl vinyl ether, or from the group of cyclic ethers consisting of tetrahydrofuran, 1,4-dioxane and morpholine

When the organic compound comprises a dilactone function, said organic compound may be chosen from the group of the cyclic dilactones having 4 ring members consisting of 1,2-dioxetanedione, or from the group of the cyclic dilactones having 5 ring members consisting of 1,3-dioxolane-4,5-dione, 1,5-dioxolane-2,4-dione, and 2,2-dibutyl-1,5-dioxolane-2,4-dione, or from the group of the cyclic dilactones having 6 ring members consisting of 1,3-dioxane-4,6-dione, 2,2-dimethyl-1,3-dioxane-4,6-dione, 2,2,5-trimethyl-1,3-dioxane-4,6-dione, 1,4-dioxane-2,5-dione, 3,6-dimethyl-1,4-dioxane-2,5-dione, 3,6-diisopropyl-1,4-dioxane-2,5-dione, and 3,3-ditoluyl-6,6-diphenyl-1,4-dioxane-2,5-dione, or from the group of the cyclic dilactones having 7 ring members consisting of 1,2-dioxepane-3,7-dione, 1,4-dioxepane-5,7-dione, 1,3-dioxepane-4,7-dione and 5-hydroxy-2,2-dimethyl-1,3-dioxepane-4,7-dione.

When the organic compound comprises a carboxyanhydride function, said organic compound may be chosen from the group of the O-carboxyanhydrides consisting of 5-methyl-1,3-dioxolane-2,4-dione and 2,5-dioxo-1,3-dioxolane-4-propanoic acid, or from the group of the N-carboxyanhydrides consisting of 2,5-oxazolidinedione and 3,4-dimethyl-2,5-oxazolidinedione. “Carboxyanhydride” is understood to mean a cyclic organic compound comprising a carboxyanhydride function, that is to say a —CO—O—CO—X— or —X—CO—O—CO— sequence within the ring, with —CO— corresponding to a carbonyl function and X being able to be an oxygen or nitrogen atom. For X═O, reference is made to an O-carboxyanhydride, and when X═N, reference is made to an N-carboxyanhydride.

The addition of the organic compound to the porous support may be carried out by two variant embodiments described in detail below.

Variant 1

According to a first embodiment according to the invention, the hydrogenation process is carried out in the presence of a catalyst obtained by a preparation process wherein step a) is carried out by simultaneously bringing together said porous support and said organic compound in the liquid state, and without physical contact, at a temperature below the boiling point of said organic compound and under conditions of pressure and duration such that a fraction of said organic compound is transferred in the gaseous state to the porous support.

In this embodiment, the process for adding the organic compound does not involve a conventional step of impregnation using a solution containing a solvent in which the organic compound is diluted. Consequently, it is not necessary to carry out a step of drying the porous support with a view to eliminating the solvent, resulting in a process that is more economical in terms of hot utility and raw material. Moreover, according to this embodiment, the step of adding the organic compound is carried out at a temperature below the boiling point of said organic compound, which affords a substantial gain from an energy point of view and in terms of safety. Indeed, for many organic compounds, such as ethylene glycol for example, the ignition point is lower than the boiling point. There is therefore a risk of fire when working at a temperature above the boiling point of the organic compound. Furthermore, a high temperature may also lead to a partial or complete decomposition of the organic compound, greatly reducing its effect. For example, citric acid, commonly used as an organic additive (US2009/0321320), decomposes at 175° C. whereas its boiling point is 368° C. at atmospheric pressure. The preparation process is also characterized by the fact that the addition of the organic compound to the porous support is carried out without physical contact with the organic compound in the liquid state, that is to say without impregnation of the porous support with the liquid. The process is based on the principle of the existence of a vapor pressure of the organic compound which is generated by its liquid phase at a given temperature and a given pressure. Thus, a portion of the molecules of organic compound in the liquid state passes into the gaseous state (vaporization) and is then transferred (gaseously) to the porous support. This bringing-together step a) is carried out for a period sufficient to attain the targeted content of organic compound in the porous solid which is used as catalyst support.

In this embodiment, the step of adding the organic compound to a porous support may be carried out in a unit for adding said organic compound. The addition unit used comprises a first compartment and a second compartment that are in communication so as to allow the passage of a gaseous fluid between the two compartments, the first compartment being suitable for containing the porous support and the second compartment being suitable for containing the organic compound in liquid form. In this embodiment, the process comprises a step a) wherein the porous support and the organic compound in liquid form are brought together without physical contact between the porous support and the organic compound in liquid form, at a temperature below the boiling point of the organic compound and under conditions of pressure and duration such that a fraction of said organic compound is transferred gaseously to the porous solid by circulation of a stream of organic compound in gaseous form from the second compartment into the first compartment, so as to ultimately provide a porous support containing the organic compound.

According to one embodiment, the addition unit comprises a chamber that includes the first and second compartments, the compartments being in gaseous communication. For example, the compartments are arranged side by side and separated by a partition, for example a substantially vertical partition, attached to the bottom of the chamber and extending only over a fraction of the height of the chamber so as to allow the gaseous overhead to diffuse from one compartment to the other. Alternatively, the compartments are arranged one on top of the other and are in communication so as to allow the passage of the organic compound in the gaseous state between the two compartments. Preferably, the chamber is closed.

According to another embodiment, the addition unit comprises two chambers that respectively form the first and second compartments, the two chambers being in gaseous communication, for example by means of a duct. Preferably, the two chambers are closed.

Preferably, the compartment intended to contain the liquid organic compound comprises means for setting said liquid in motion in order to facilitate the transfer of the organic compound in the gaseous state from one compartment to the other. According to one preferred embodiment, the two compartments comprise means for respectively setting the liquid and the porous support in motion. Advantageously, the compartment containing the organic compound in the liquid state is equipped with internals intended to maximize the surface area of the gas/liquid interface. These internals are for example porous monoliths impregnated by capillary action, falling films, packings or any other means known to those skilled in the art.

In a preferred embodiment, step a) is carried out in the presence of a (carrier) gas circulating from the second compartment into the first compartment so as to entrain the organic molecules in the gaseous state into the compartment containing the porous support. For example, the carrier gas may be chosen from carbon dioxide, ammonia, air with a controlled moisture content, an inert gas such as argon, nitrogen, hydrogen, natural gas or a refrigerant gas according to the classification published by IUPAC.

According to a preferred embodiment, step a) comprises a step wherein a gaseous effluent containing said organic compound is withdrawn from the first compartment and the effluent is recycled to the first and/or the second compartment.

According to another embodiment, a gaseous effluent containing said organic compound in the gaseous state is withdrawn from the first compartment, said effluent is condensed so as to recover a liquid fraction containing the organic compound in the liquid state, and said liquid fraction is recycled to the second compartment.

Step a) is preferably carried out at an absolute pressure of between 0.1 and 1 MPa. As specified above, the temperature of step a) is set at a temperature below the boiling point of the organic compound. The temperature of step a) is generally below 200° C., preferably between 10° C. and 150° C., more preferably between 25° C. and 120° C.

Variant 2

According to a second embodiment according to the invention, the hydrogenation process is carried out in the presence of a catalyst obtained by a preparation process wherein step a) is carried out by bringing together said porous support with a porous solid (also referred to herein as “carrier solid”) comprising said organic compound under conditions of temperature, pressure and duration such that a fraction of said organic compound is transferred gaseously from said carrier solid to said porous support.

The aim of this bringing-together of the porous support and the carrier solid comprising the organic compound is to enable a gaseous transfer of a portion of the organic compound contained in the carrier solid to the porous support. This step is based on the principle of the existence of a vapor pressure of the organic compound at a given temperature and a given pressure. Thus, a portion of the molecules of organic compound of the carrier solid comprising the organic compound passes into gaseous form (vaporization) and is then transferred (gaseously) to the porous support. According to this embodiment, the porous solid (“carrier solid”) serves as a source of organic compound to enrich, in organic compound, the porous support, which preferably does not initially comprise organic compound. This embodiment is therefore different than a simple maturation step as conventionally encountered in the prior art. Indeed, the diffusion of the organic compound from the carrier solid toward the porous support is carried out in condensed form inside each grain of the solid (intergranularly), unlike a conventional maturation for which the diffusion of the organic compound is carried out intragranularly (inside each grain of the support). Such a definition of maturation is illustrated in the thesis by Jonathan Moreau, “Rationalisation de l'étape d'imprégnation de catalyseurs à base d'hétéropolyanions de molybdène supportés sur alumine” [Rationalization of the step of impregnation of catalysts based on molybdenum heteropolyanions supported on alumina]; page 56; University Claude Bernard—Lyon 1, 2012.

Moreover, the use of such a step of contacting, i.e. by gaseous transfer, between the porous solid comprising the organic compound and the porous support can make it possible to save on a drying step which would conventionally have taken place after a step of impregnation of the organic compound diluted in a solvent on the porous support (optionally followed by a maturation step) in order to eliminate the solvent used. Indeed, in this embodiment, the porous solid (“carrier solid”) comprising the organic compound is obtained by impregnation with the organic compound in the liquid state. Unlike the prior art, the organic compound is not diluted in a solvent. One advantage of this embodiment compared to the prior art processes therefore lies in the absence of a drying step which is conventionally used for eliminating the solvent after the impregnation step and therefore of being less energy-consuming compared to conventional processes. This absence of a drying step can make it possible to avoid any losses of organic compound by vaporization or even by degradation.

The volume of organic compound used is strictly less than the total volume of the accessible porosity of the porous solid and of the porous support used in step a) and is set relative to the targeted amount of organic compound on the porous solid at the end of step a). Another advantage of this embodiment is therefore the use of a smaller amount of organic compound relative to the case of the prior art where, in the absence of solvent, the entire porosity would have to be filled with organic compound.

The weight ratio of (porous solid comprising the organic compound)/(porous support) depends on the pore distribution of the porous solid and the porous support and on the aim in terms of targeted amount of organic compound on the porous support. This weight ratio is generally less than or equal to 10, preferably less than 2 and even more preferably between 0.05 and 1, limits included.

In this embodiment, step a) is carried out under conditions of temperature, pressure and duration so as to achieve a balance between the amount of organic compound on the porous solid (“carrier solid”) and the porous support. The term “balance” is understood to denote the fact that at the end of step a), at least 50% by weight of the porous solid and the porous support have an amount of said organic compound equal to plus or minus 50% of the targeted amount, preferably at least 80% by weight of the porous solid and the porous support have an amount of said organic compound equal to plus or minus 40% of the targeted amount and more preferentially still at least 90% by weight of the porous solid and the porous support have an amount of said organic compound equal to plus or minus 20% of the targeted amount.

By way of nonlimiting example, in the case in which the preparation of a porous support comprising 5% by weight of organic compound is targeted, it is possible to bring together, in a same amount, a porous solid containing 10% by weight of organic compound with the porous support free of said organic compound. It will be considered in this case that the balance is achieved when at least 50% by weight of the porous solid and the porous support have an amount of said organic compound which corresponds to a content of between 2.5% and 7.5% by weight, preferentially when at least 80% by weight of the porous solid and the porous support have an amount of said organic compound which corresponds to a content which is between 3% and 7% by weight, and more preferentially still when at least 90% by weight of the porous solid and the porous support have an amount of said organic compound which corresponds to a content of between 4% and 6% by weight.

These contents may be determined by a statistically representative sampling for which the samples may be characterized for example by assaying of the carbon and/or possible heteroatoms contained in the organic compound or by thermogravimetry coupled to an analyzer, for example a mass spectrometer, or an infrared spectrometer and thus determine the respective contents of organic compounds.

Step a) is preferably carried out under controlled temperature and pressure conditions and so that the temperature is below the boiling point of said organic compound to be transferred gaseously.

Preferably, the operating temperature is below 150° C. and the absolute pressure is generally between 0.1 and 1 MPa, preferably between 0.1 and 0.5 MPa and more preferably between 0.1 and 0.2 MPa. It is thus possible to perform the bringing-together step in an open or closed chamber, optionally with control of the composition of the gas present in the chamber.

When the step of bringing together the porous solid and the porous support is carried out in an open chamber, it will be ensured that the entrainment of the organic compound out of the chamber is limited as much as possible. Alternatively, the step of bringing together the porous solid and the porous support may be carried out in a closed chamber, for example in a container for storing or transporting the solid that is impermeable to gas exchanges with the outside environment. In this embodiment, the bringing-together step may be carried out by controlling the composition of the gas forming the atmosphere by introducing one or more gaseous compounds optionally with a controlled moisture content. By way of nonlimiting example, the gaseous compound may be carbon dioxide, ammonia, air with a controlled moisture content, an inert gas such as argon, nitrogen, hydrogen, natural gas or a refrigerant gas according to the classification published by IUPAC. According to one advantageous embodiment, the step of bringing together in a controlled gaseous atmosphere uses a forced circulation of the gas in the chamber.

In one embodiment of this variant embodiment, the step of bringing together the porous solid and the porous support is carried out without physical contact, in a chamber equipped with compartments suitable for containing, respectively, the porous solid (“carrier solid”) and the porous support, the compartments being in communication so as to allow the passage of the organic compound in the gaseous state between the two compartments. It is advantageous to circulate a gas stream firstly through the compartment containing the porous solid comprising the organic compound then through the compartment containing the porous support.

Preferably, the porous solid (“carrier solid”) is of a different nature than the porous solid (serving as catalyst support); that is to say that the porous solid has at least one distinguishing physical feature with regard to the porous support in order to enable for example the subsequent separation thereof. For example and nonlimitingly, this physical feature may be:
the size of the particles of the solid: the separation can be carried out on a sieve or by cyclone;
magnetism: the separation is carried out by the application of a magnetic field;
the density of the solid: optionally in conjunction with the size of the particles, this difference in density can for example be used for separation by elutriation or by cyclone;
the dielectric constant: the separation takes place by application of an electrostatic field.

Moreover, said porous support and said porous solid containing the organic compound may advantageously be of different porosity and/or different chemical nature. Indeed, the porous solid may be of a suitable chemical composition to restrict adsorption of the compound to be impregnated compared to the adsorption of the compound to be impregnated on the porous support. A similar effect may be obtained by adapting the porous structure of the porous solid so that it has a mean pore opening that is greater than that of the porous support so as to favor the transfer of the organic compound to the porous support, particularly in the case of capillary condensation.

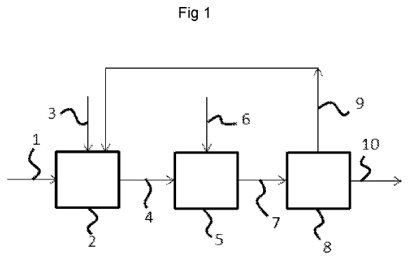

One embodiment of step a) of bringing the organic compound and the porous support together is illustrated schematically in FIG. 1. This embodiment according to the invention corresponds to the case in which the porous solid containing the organic compound acts as a reservoir of organic compound for the porous support. As indicated in FIG. 1, a “carrier” porous solid 1 is impregnated in an impregnation unit 2 with a liquid organic compound supplied by the line 3. The carrier solid 4 comprising the organic compound is transferred into the addition unit 5 in which said carrier solid is brought together with the porous support supplied by the line 6. At the end of the step of bringing the porous solid and the porous support together, a mixture of porous support and porous solid (carrier solid), each containing said organic compound, is withdrawn from the unit by the line 7. The mixture of solids (porous support and porous solid) is then sent to a separation unit 8 which carries out a physical separation of the solids (porous solid and porous support). Owing to the use of the separation, two streams of solids are obtained, namely the porous solid 9 containing the organic compound and the porous support 10 also containing the organic compound. In accordance with this embodiment, the porous solid still containing the organic compound 9 is recycled to the unit for introducing the liquid organic compound with a view to a subsequent use.

Step b)

Step b) of bringing said porous support into contact with at least one solution containing at least one salt of a precursor of the phase comprising at least one group VIII metal may be carried out by dry impregnation or excess impregnation according to methods well known to those skilled in the art. Said step b) is preferentially carried out by bringing the porous support into contact with at least one solution, which is aqueous or organic (for example methanol or ethanol or phenol or acetone or toluene or dimethyl sulfoxide (DMSO)) or else consists of a mixture of water and of at least one organic solvent, containing at least one precursor of the active phase comprising at least one group VIII metal at least partially in the dissolved state, or else in bringing a precursor of the active phase into contact with at least one colloidal solution of at least one group VIII metal precursor, in the oxidized form (nanoparticles of nickel oxides, of nickel oxy(hydroxide) or of nickel hydroxide) or in the reduced form (metal nanoparticles of the group VIII metal in the reduced state). Preferably, the solution is aqueous. The pH of this solution may be modified by the optional addition of an acid or of a base. According to another preferred alternative form, the aqueous solution may contain ammonia or ammonium NH₄⁺ ions.

Preferably, said step b) is carried out by dry impregnation, which consists in bringing the porous support into contact with at least one solution containing at least one precursor of the active phase comprising at least one group VIII metal, of which the volume of the solution is between 0.25 and 1.5 times the pore volume of the support of the catalyst precursor to be impregnated.

Preferably, the group VIII metal is chosen from nickel, palladium or platinum. More preferentially, the group VIII metal is nickel.

When the precursor of the active phase is introduced in aqueous solution and when the group VIII metal is nickel, use is advantageously made of a precursor of nickel in the form of nitrate, carbonate, chloride, sulfate, hydroxide, hydroxycarbonate, formate, acetate, oxalate, of complexes formed with acetylacetonates, or also of tetrammine or hexammine complexes, or of any other inorganic derivative soluble in aqueous solution, which is brought into contact with said catalyst precursor. Use is advantageously made, as nickel precursor, of nickel nitrate, nickel carbonate, nickel chloride, nickel hydroxide or nickel hydroxycarbonate. Very preferably, the nickel precursor is nickel nitrate, nickel carbonate or nickel hydroxide.

The nickel content is between 1 and 65% by weight of said element relative to the total weight of the catalyst, preferably between 5 and 55% by weight, even more preferably between 8 and 40% by weight, and particularly preferred between 12 and 35% by weight. The Ni content is measured by X-ray fluorescence.

When it is desired to use the catalyst according to the invention in a reaction for the selective hydrogenation of polyunsaturated molecules such as diolefins, acetylenics or alkenylaromatics, the nickel content is advantageously between 1 and 35% by weight, preferably between 5 and 30% by weight, and more preferentially between 8 and 25% by weight, and even more preferably between 12 and 23% by weight of said element relative to the total weight of the catalyst.

When it is desired to use the catalyst according to the invention in an aromatics hydrogenation reaction, the nickel content is advantageously between 8 and 65% by weight, preferably between 12 and 55% by weight, even more preferably between 15 and 40% by weight, and more preferentially between 18 and 35% by weight of said element relative to the total weight of the catalyst.

Advantageously, the molar ratio of said organic compound introduced in step a) to the group VIII metal introduced in step b) is between 0.01 and 5.0 mol/mol, preferably between 0.05 and 2.0 mol/mol, more preferentially between 0.1 and 1.5 mol/mol and more preferentially still between 0.3 and 1.2 mol/mol, relative to the group VIII element.

Step c) Drying

The drying step c) is carried out at a temperature of less than 250° C., preferably greater than 15° C. and less than 250° C., more preferentially between 30 and 220° C., even more preferentially between 50 and 200° C., and even more preferentially between 70 and 180° C., for a period of time typically of between 10 minutes and 24 hours. Longer periods of time are not ruled out, but do not necessarily afford any improvement.

The drying step can be carried out by any technique known to those skilled in the art. It is advantageously carried out under an inert atmosphere or under an oxygen-containing atmosphere or under a mixture of inert gas and oxygen. It is advantageously carried out at atmospheric pressure or at reduced pressure. Preferably, this step is carried out at atmospheric pressure and in the presence of air or nitrogen.

Step d) Calcination (Optional)

Optionally, at the end of the sequence of steps a), b) and c), and indifferently according to the order of the sequence of these steps (as described above), a calcination step d) is carried out at a temperature of between 250° C. and 1000° C., preferably of between 250° C. and 750° C., under an inert atmosphere or under an oxygen-containing atmosphere. The duration of this heat treatment is generally between 15 minutes and 10 hours. Longer periods of time are not ruled out, but do not necessarily provide any improvement. After this treatment, the nickel of the active phase is thus in oxide form and the catalyst contains no more or very little organic compound introduced during the synthesis thereof. However, the introduction of the organic compound during the preparation thereof has made it possible to increase the dispersion of the active phase thus leading to a more active and/or more selective catalyst.

Step e) Reduction by a Reducing Gas (Optional)

Prior to the use of the catalyst in the catalytic reactor and the implementation of a hydrogenation process, at least one reducing treatment step e) is advantageously carried out in the presence of a reducing gas after the sequence of steps a), b) and c), optionally d), and indifferently according to the order of the sequence of these steps (as described above), so as to obtain a catalyst comprising the group VIII metal at least partially in metallic form.

This treatment makes it possible to activate said catalyst and to form metal particles, in particular of nickel in the zero-valent state. Said reducing treatment may be carried out in situ or ex situ, that is to say after or before the catalyst is charged to the hydrogenation reactor. Said reduction step e) can be carried out on the catalyst which may or may not have been subjected to the passivation step f), described below.

The reducing gas is preferably hydrogen. The hydrogen can be used pure or as a mixture (for example a hydrogen/nitrogen, hydrogen/argon or hydrogen/methane mixture). In the case where the hydrogen is used as a mixture, all proportions can be envisaged.

Said reducing treatment is carried out at a temperature of between 120 and 500° C., preferably between 150 and 450° C. When the catalyst is not subjected to passivation, or is subjected to a reducing treatment before passivation, the reducing treatment is carried out at a temperature of between 350 and 500° C., preferably between 350 and 450° C. When the catalyst has been subjected beforehand to a passivation, the reducing treatment is generally carried out at a temperature of between 120 and 350° C., preferably between 150 and 350° C.

The duration of the reducing treatment is generally between 2 and 40 hours, preferably between 3 and 30 hours. The rise in temperature up to the desired reduction temperature is generally slow, for example set between 0.1 and 10° C./min, preferably between 0.3 and 7° C./min.

The hydrogen flow rate, expressed in 1/hour/gram of catalyst, is between 0.1 and 100 l/hour/gram of catalyst, preferably between 0.5 and 10 l/hour/gram of catalyst, even more preferably between 0.7 and 5 l/hour/gram of catalyst.

Step f) Passivation (Optional)

Prior to its use in the catalytic reactor, the catalyst according to the invention may optionally undergo a passivation step (step f) with a sulfur-containing or oxygen-containing compound or with CO₂, before or after the reducing treatment step e). This passivation step can be carried out ex situ or in situ. The passivation step is carried out by the use of methods known to those skilled in the art.

The step of passivation by sulfur makes it possible to improve the selectivity of the catalysts and to prevent thermal runaways during the start-ups of fresh catalysts. The passivation generally consists in irreversibly poisoning, by the sulfur-containing compound, the most virulent active sites of the nickel which exist on the fresh catalyst and thus in weakening the activity of the catalyst in favor of its selectivity. The passivation step is carried out by the use of methods known to those skilled in the art and in particular, by way of example, by the use of one of the methods described in the patent documents EP0466567, U.S. Pat. No. 5,153,163, FR2676184, WO2004/098774 and EP0707890. The sulfur-containing compound is, for example, chosen from the following compounds: thiophene, thiophane, alkyl monosulfides, such as dimethyl sulfide, diethyl sulfide, dipropyl sulfide and propyl methyl sulfide, or also an organic disulfide of formula HO—R₁—S—S—R₂—OH, such as dithiodiethanol of formula HO—C₂H₄—S—S—C₂H₄—OH (often referred to as DEODS). The sulfur content is generally between 0.1% and 2% by weight of said element with respect to the weight of the catalyst.

The step of passivation by an oxygen-containing compound or by CO₂ is generally carried out after a reducing treatment beforehand at high temperature, generally of between 350 and 500° C., and makes it possible to preserve the metallic phase of the catalyst in the presence of air. A second reducing treatment at lower temperature, generally between 120 and 350° C., is subsequently generally carried out. The oxygen-containing compound is generally air or any other oxygen-containing stream.

Characteristics of the Catalyst

The catalyst obtained using the preparation process comprises a porous support and an active phase comprising, preferably consisting of, at least one group VIII metal, preferably nickel, palladium or platinum, more preferentially nickel, said active phase not comprising a group VIB metal. In particular, it does not comprise molybdenum or tungsten.

When the metal is nickel, the nickel content is between 1 and 65% by weight of said element relative to the total weight of the catalyst, preferably between 5 and 55% by weight, even more preferably between 8 and 40% by weight, and particularly preferably between 12 and 35% by weight. When it is desired to use the catalyst according to the invention in a reaction for the selective hydrogenation of polyunsaturated molecules such as diolefins, acetylenics or alkenylaromatics, the nickel content is advantageously between 1 and 35% by weight, preferably between 5 and 30% by weight, and more preferentially between 8 and 25% by weight, and even more preferably between 12 and 23% by weight of said element relative to the total weight of the catalyst.

When it is desired to use the catalyst according to the invention in an aromatics hydrogenation reaction, the nickel content is advantageously between 8 and 65% by weight, preferably between 12 and 55% by weight, even more preferably between 15 and 40% by weight, and more preferentially between 18 and 35% by weight of said element relative to the total weight of the catalyst.

The active phase is in the form of nickel particles having a diameter of less than or equal to 18 nm, said catalyst comprising a total pore volume, measured by mercury porosimetry, of between 0.01 and 1.0 ml/g, a mesopore volume, measured by mercury porosimetry, of greater than 0.01 ml/g, a macropore volume, measured by mercury porosimetry, of less than or equal to 0.6 ml/g, a mesopore median diameter by volume of between 3 and 25 nm, a macropore median diameter by volume of between 50 and 1000 nm, and an SBET specific surface area of between 25 and 350 m²/g.

The size of the nickel particles in the catalyst according to the invention is less than 18 nm, preferably less than 15 nm, more preferentially between 0.5 and 12 nm, preferably between 1.5 and 8.0 nm.

The porous support on which said active phase is deposited comprises alumina (Al₂O₃). Preferably, the alumina present in said support is a transition alumina, such as a γ-, δ-, θ-, χ-, ρ- or η-alumina, alone or as a mixture. More preferably, the alumina is a γ-, δ- or θ-transition alumina, alone or as a mixture.

In a second implementation variant, the alumina present in said support is an α-alumina.

The support can comprise another oxide other than alumina, such as silica (SiO₂), titanium dioxide (TiO₂), ceria (CeO₂), zirconia (ZrO₂), or P₂O₅. The support may be a silica-alumina. Very preferably, said support consists solely of alumina.

Said catalyst is generally presented in all the forms known to those skilled in the art, for example in the form of beads (generally having a diameter of between 1 and 8 mm), of extrudates, of blocks or of hollow cylinders. Preferably, it consists of extrudates with a diameter generally of between 0.5 and 10 mm, preferably between 0.8 and 3.2 mm and very preferably between 1.0 and 2.5 mm and with a mean length of between 0.5 and 20 mm. The term “mean diameter” of the extrudates is intended to mean the mean diameter of the circle circumscribed in the cross section of these extrudates. The catalyst can advantageously be presented in the form of cylindrical, multilobal, trilobal or quadrilobal extrudates. Preferably, its shape will be trilobal or quadrilobal. The shape of the lobes could be adjusted according to all the methods known from the prior art.

The pore volume of the support is generally between 0.1 cm³/g and 1.5 cm³/g, preferably between 0.5 cm³/g and 1.0 cm³/g. The specific surface area of the support is generally greater than or equal to 5 m²/g, preferably greater than or equal to 30 m²/g, more preferentially between 40 m²/g and 500 m²/g, and more preferentially still between 50 m²/g and 400 m²/g.

When it is desired to use the catalyst according to the invention in a reaction for the selective hydrogenation of polyunsaturated molecules such as diolefins, acetylenics or alkenylaromatics, the specific surface area of the support is advantageously between 40 and 250 m²/g, preferably between 50 and 200 m²/g.

When it is desired to use the catalyst according to the invention in an aromatics hydrogenation reaction, the specific surface area of the support is advantageously between 60 and 500 m²/g, preferably between 100 and 400 m²/g.

Description of the Process for the Selective Hydrogenation of Polyunsaturated Compounds

Another subject of the present invention is a process for the selective hydrogenation of polyunsaturated compounds containing at least 2 carbon atoms per molecule, such as diolefins and/or acetylenics and/or alkenylaromatics, also known as styrenics, contained in a hydrocarbon feedstock having a final boiling point of less than or equal to 300° C., which process being carried out at a temperature of between 0 and 300° C., at a pressure of between 0.1 and 10 MPa, at a hydrogen/(polyunsaturated compounds to be hydrogenated) molar ratio of between 0.1 and 10 and at an hourly space velocity of between 0.1 and 200 h⁻¹ when the process is carried out in the liquid phase, or at a hydrogen/(polyunsaturated compounds to be hydrogenated) molar ratio of between 0.5 and 1000 and at an hourly space velocity of between 100 and 40 000 h⁻¹ when the process is carried out in the gas phase, in the presence of a catalyst obtained by the preparation process as described above in the description.

Monounsaturated organic compounds, such as, for example, ethylene and propylene, are at the root of the manufacture of polymers, of plastics and of other chemicals having added value. These compounds are obtained from natural gas, from naphtha or from gas oil which have been treated by steam cracking or catalytic cracking processes. These processes are carried out at high temperature and produce, in addition to the desired monounsaturated compounds, polyunsaturated organic compounds, such as acetylene, propadiene and methylacetylene (or propyne), 1,2-butadiene and 1,3-butadiene, vinylacetylene and ethylacetylene, and other polyunsaturated compounds, the boiling point of which corresponds to the C5+ fraction (hydrocarbon-based compounds having at least 5 carbon atoms), in particular diolefinic or styrene or indene compounds. These polyunsaturated compounds are highly reactive and result in side reactions in the polymerization units. It is thus necessary to remove them before making economic use of these fractions.

Selective hydrogenation is the main treatment developed to specifically remove undesirable polyunsaturated compounds from these hydrocarbon feedstocks. It makes possible the conversion of polyunsaturated compounds to the corresponding alkenes or aromatics while avoiding their complete saturation and thus the formation of the corresponding alkanes or naphthenes. In the case of steam cracking gasolines used as feedstock, the selective hydrogenation also makes it possible to selectively hydrogenate the alkenylaromatics to give aromatics while avoiding the hydrogenation of the aromatic nuclei.

The hydrocarbon feedstock treated in the selective hydrogenation process has a final boiling point of less than or equal to 300° C. and contains at least 2 carbon atoms per molecule and comprises at least one polyunsaturated compound. The term “polyunsaturated compounds” is intended to mean compounds comprising at least one acetylenic function and/or at least one diene function and/or at least one alkenylaromatic function.

More particularly, the feedstock is selected from the group consisting of a steam cracking C2 fraction, a steam cracking C2-C3 fraction, a steam cracking C3 fraction, a steam cracking C4 fraction, a steam cracking C5 fraction and a steam cracking gasoline, also known as pyrolysis gasoline or C5+ fraction.

The steam cracking C2 fraction, advantageously used for the implementation of the selective hydrogenation process according to the invention, exhibits, for example, the following composition: between 40% and 95% by weight of ethylene and of the order of 0.1% to 5% by weight of acetylene, the remainder being essentially ethane and methane. In some steam cracking C2 fractions, between 0.1% and 1% by weight of C3 compounds may also be present.

The steam cracking C3 fraction, advantageously used for the implementation of the selective hydrogenation process according to the invention, exhibits, for example, the following mean composition: of the order of 90% by weight of propylene and of the order of 1% to 8% by weight of propadiene and of methylacetylene, the remainder being essentially propane. In some C3 fractions, between 0.1% and 2% by weight of C2 compounds and of C4 compounds may also be present.

A C2-C3 fraction can also advantageously be used for the implementation of the selective hydrogenation process according to the invention. It exhibits, for example, the following composition: of the order of 0.1% to 5% by weight of acetylene, of the order of 0.1% to 3% by weight of propadiene and of methylacetylene, of the order of 30% by weight of ethylene and of the order of 5% by weight of propylene, the remainder being essentially methane, ethane and propane. This feedstock may also contain between 0.1% and 2% by weight of C4 compounds.

The steam cracking C4 fraction, advantageously used for the implementation of the selective hydrogenation process according to the invention, exhibits, for example, the following mean composition by weight: 1% by weight of butane, 46.5% by weight of butene, 51% by weight of butadiene, 1.3% by weight of vinylacetylene and 0.2% by weight of butyne. In some C4 fractions, between 0.1% and 2% by weight of C3 compounds and of C5 compounds may also be present.

The steam cracking C5 fraction, advantageously used for the implementation of the selective hydrogenation process according to the invention, exhibits, for example, the following composition: 21% by weight of pentanes, 45% by weight of pentenes and 34% by weight of pentadienes.

The steam cracking gasoline or pyrolysis gasoline, advantageously used for the implementation of the selective hydrogenation process according to the invention, corresponds to a hydrocarbon-based fraction, the boiling point of which is generally between 0 and 300° C., preferably between 10 and 250° C. The polyunsaturated hydrocarbons to be hydrogenated present in said steam cracking gasoline are in particular diolefin compounds (butadiene, isoprene, cyclopentadiene, and the like), styrene compounds (styrene, α-methylstyrene, and the like) and indene compounds (indene, and the like). The steam cracking gasoline generally comprises the C5-C12 fraction with traces of C3, C4, C13, C14 and C15 (for example between 0.1% and 3% by weight for each of these fractions). For example, a feedstock formed of pyrolysis gasoline generally has a composition as follows: 5% to 30% by weight of saturated compounds (paraffins and naphthenes), 40% to 80% by weight of aromatic compounds, 5% to 20% by weight of mono-olefins, 5% to 40% by weight of diolefins and 1% to 20% by weight of alkenylaromatic compounds, the combined compounds forming 100%. It also contains from 0 to 1000 ppm by weight of sulfur, preferably from 0 to 500 ppm by weight of sulfur.

Preferably, the polyunsaturated hydrocarbon feedstock treated in accordance with the selective hydrogenation process according to the invention is a steam cracking C2 fraction or a steam cracking C2-C3 fraction or a steam cracking gasoline.

The selective hydrogenation process according to the invention is targeted at removing said polyunsaturated hydrocarbons present in said feedstock to be hydrogenated without hydrogenating the monounsaturated hydrocarbons. For example, when said feedstock is a C2 fraction, the selective hydrogenation process is targeted at selectively hydrogenating acetylene. When said feedstock is a C3 fraction, the selective hydrogenation process is targeted at selectively hydrogenating propadiene and methylacetylene. In the case of a C4 fraction, the aim is to remove butadiene, vinylacetylene (VAC) and butyne; in the case of a C5 fraction, the aim is to remove the pentadienes. When said feedstock is a steam cracking gasoline, the selective hydrogenation process is targeted at selectively hydrogenating said polyunsaturated hydrocarbons present in said feedstock to be treated so that the diolefin compounds are partially hydrogenated to give mono-olefins and so that the styrene and indene compounds are partially hydrogenated to give corresponding aromatic compounds while avoiding the hydrogenation of the aromatic nuclei.

The technological implementation of the selective hydrogenation process is, for example, carried out by injection, as ascending or descending stream, of the polyunsaturated hydrocarbon feedstock and of the hydrogen into at least one fixed bed reactor. Said reactor may be of isothermal type or of adiabatic type. An adiabatic reactor is preferred. The polyunsaturated hydrocarbon feedstock can advantageously be diluted by one or more reinjection(s) of the effluent, resulting from said reactor where the selective hydrogenation reaction takes place, at various points of the reactor, located between the inlet and the outlet of the reactor, in order to limit the temperature gradient in the reactor. The technological implementation of the selective hydrogenation process according to the invention can also advantageously be carried out by the implantation of at least said supported catalyst in a reactive distillation column or in reactors-exchangers or in a slurry-type reactor. The stream of hydrogen may be introduced at the same time as the feedstock to be hydrogenated and/or at one or more different points of the reactor.

The selective hydrogenation of the steam cracking C2, C2-C3, C3, C4, C5 and C5+ fractions can be carried out in the gas phase or in the liquid phase, preferably in the liquid phase for the C3, C4, C5 and C5+ fractions and in the gas phase for the C2 and C2-C3 fractions. A liquid-phase reaction makes it possible to lower the energy cost and to increase the cycle period of the catalyst.

Generally, the selective hydrogenation of a hydrocarbon feedstock containing polyunsaturated compounds containing at least 2 carbon atoms per molecule and having a final boiling point of less than or equal to 300° C. is carried out at a temperature of between 0 and 300° C., at a pressure of between 0.1 and 10 MPa, at a hydrogen/(polyunsaturated compounds to be hydrogenated) molar ratio of between 0.1 and 10 and at an hourly space velocity HSV (defined as the ratio of the flow rate by volume of feedstock to the volume of the catalyst) of between 0.1 and 200 h⁻¹ for a process carried out in the liquid phase, or at a hydrogen/(polyunsaturated compounds to be hydrogenated) molar ratio of between 0.5 and 1000 and at an hourly space velocity HSV of between 100 and 40 000 h⁻¹ for a process carried out in the gas phase.

In one embodiment according to the invention, when a selective hydrogenation process is carried out wherein the feedstock is a steam cracking gasoline comprising polyunsaturated compounds, the (hydrogen)/(polyunsaturated compounds to be hydrogenated) molar ratio is generally between 0.5 and 10, preferably between 0.7 and 5.0 and more preferably still between 1.0 and 2.0, the temperature is between 0 and 200° C., preferably between 20 and 200° C. and more preferably still between 30 and 180° C., the hourly space velocity (HSV) is generally between 0.5 and 100 h⁻¹, preferably between 1 and 50 h⁻¹, and the pressure is generally between 0.3 and 8.0 MPa, preferably between 1.0 and 7.0 MPa and more preferably still between 1.5 and 4.0 MPa.

More preferentially, a selective hydrogenation process is carried out wherein the feedstock is a steam cracking gasoline comprising polyunsaturated compounds, the hydrogen/(polyunsaturated compounds to be hydrogenated) molar ratio is between 0.7 and 5.0, the temperature is between 20 and 200° C., the hourly space velocity (HSV) is generally between 1 and 50 h⁻¹ and the pressure is between 1.0 and 7.0 MPa.

More preferentially still, a selective hydrogenation process is carried out wherein the feedstock is a steam cracking gasoline comprising polyunsaturated compounds, the hydrogen/(polyunsaturated compounds to be hydrogenated) molar ratio is between 1.0 and 2.0, the temperature is between 30 and 180° C., the hourly space velocity (HSV) is generally between 1 and 50 h⁻¹ and the pressure is between 1.5 and 4.0 MPa.

The hydrogen flow rate is adjusted in order to have available a sufficient amount thereof to theoretically hydrogenate all of the polyunsaturated compounds and to maintain an excess of hydrogen at the reactor outlet.

In another embodiment according to the invention, when a selective hydrogenation process is carried out wherein the feedstock is a steam cracking C2 fraction and/or a steam cracking C2-C3 fraction comprising polyunsaturated compounds, the (hydrogen)/(polyunsaturated compounds to be hydrogenated) molar ratio is generally between 0.5 and 1000, preferably between 0.7 and 800, the temperature is between 0 and 300° C., preferably between 15 and 280° C., the hourly space velocity (HSV) is generally between 100 and 40 000 h⁻¹, preferably between 500 and 30 000 h⁻¹, and the pressure is generally between 0.1 and 6.0 MPa, preferably between 0.2 and 5.0 MPa.

Description of the Process for the Hydrogenation of the Aromatics

Another subject of the present invention is a process for the hydrogenation of at least one aromatic or polyaromatic compound contained in a hydrocarbon feedstock having a final boiling point of less than or equal to 650° C., generally between 20 and 650° C., and preferably between 20 and 450° C. Said hydrocarbon feedstock containing at least one aromatic or polyaromatic compound can be chosen from the following petroleum or petrochemical fractions: the reformate from catalytic reforming, kerosene, light gas oil, heavy gas oil, cracking distillates, such as FCC recycle oil, coking unit gas oil or hydrocracking distillates.

The content of aromatic or polyaromatic compounds contained in the hydrocarbon feedstock treated in the hydrogenation process according to the invention is generally between 0.1 and 80% by weight, preferably between 1 and 50% by weight, and particularly preferably between 2 and 35% by weight, the percentage being based on the total weight of the hydrocarbon feedstock. The aromatic compounds present in said hydrocarbon feedstock are, for example, benzene or alkylaromatics, such as toluene, ethylbenzene, o-xylene, m-xylene or p-xylene, or also aromatics having several aromatic rings (polyaromatics), such as naphthalene.

The sulfur or chlorine content of the feedstock is generally less than 5000 ppm by weight of sulfur or chlorine, preferably less than 100 ppm by weight, and particularly preferably less than 10 ppm by weight.

The technological implementation of the process for the hydrogenation of aromatic or polyaromatic compounds is, for example, carried out by injection, as ascending or descending stream, of the hydrocarbon feedstock and of the hydrogen into at least one fixed bed reactor.

Said reactor may be of isothermal type or of adiabatic type. An adiabatic reactor is preferred. The hydrocarbon feedstock may advantageously be diluted by one or more reinjection(s) of the effluent, resulting from said reactor where the reaction for the hydrogenation of the aromatics takes place, at various points of the reactor, located between the inlet and the outlet of the reactor, in order to limit the temperature gradient in the reactor. The technological implementation of the process for the hydrogenation of the aromatics according to the invention may also advantageously be carried out by the implantation of at least said supported catalyst in a reactive distillation column or in reactors-exchangers or in a slurry-type reactor. The stream of hydrogen may be introduced at the same time as the feedstock to be hydrogenated and/or at one or more different points of the reactor.

The hydrogenation of the aromatic or polyaromatic compounds may be carried out in the gas phase or in the liquid phase, preferably in the liquid phase. Generally, the hydrogenation of the aromatic or polyaromatic compounds is carried out at a temperature of between 30 and 350° C., preferably between 50 and 325° C., at a pressure of between 0.1 and 20 MPa, preferably between 0.5 and 10 MPa, at a hydrogen/(aromatic compounds to be hydrogenated) molar ratio between 0.1 and 10 and at an hourly space velocity HSV of between 0.05 and 50 h⁻¹, preferably between 0.1 and 10 h⁻¹, of a hydrocarbon feedstock containing aromatic or polyaromatic compounds and having a final boiling point less than or equal to 650° C., generally between 20 and 650° C., and preferably between 20 and 450° C.

The hydrogen flow rate is adjusted in order to have available a sufficient amount thereof to theoretically hydrogenate all of the aromatic compounds and to maintain an excess of hydrogen at the reactor outlet.

The conversion of the aromatic or polyaromatic compounds is generally greater than 20 mol %, preferably greater than 40 mol %, more preferably greater than 80 mol %, and particularly preferably greater than 90 mol % of the aromatic or polyaromatic compounds contained in the hydrocarbon-based feedstock. The conversion is calculated by dividing the difference between the total moles of the aromatic or polyaromatic compounds in the hydrocarbon feedstock and in the product by the total moles of the aromatic or polyaromatic compounds in the hydrocarbon feedstock.

According to a specific alternative form of the process according to the invention, a process for the hydrogenation of the benzene of a hydrocarbon feedstock, such as the reformate resulting from a catalytic reforming unit, is carried out. The benzene content in said hydrocarbon feedstock is generally between 0.1 and 40% by weight, preferably between 0.5 and 35% by weight, and particularly preferably between 2 and 30% by weight, the percentage by weight being based on the total weight of the hydrocarbon feedstock.

The sulfur or chlorine content of the feedstock is generally less than 10 ppm by weight of sulfur or chlorine respectively, and preferably less than 2 ppm by weight.

The hydrogenation of the benzene contained in the hydrocarbon feedstock may be carried out in the gas phase or in the liquid phase, preferably in the liquid phase. When it is carried out in the liquid phase, a solvent may be present, such as cyclohexane, heptane or octane. Generally, the hydrogenation of the benzene is carried out at a temperature of between 30 and 250° C., preferably between 50 and 200° C., and more preferably between 80 and 180° C., at a pressure of between 0.1 and 10 MPa, preferably between 0.5 and 4 MPa, at a hydrogen/(benzene) molar ratio between 0.1 and 10 and at an hourly space velocity HSV of between 0.05 and 50 h⁻¹, preferably between 0.5 and 10 h⁻¹.

The conversion of the benzene is generally greater than 50 mol %, preferably greater than 80 mol %, more preferably greater than 90 mol % and particularly preferably greater than 98 mol %.

### EXAMPLES

The following examples specify the advantage of the invention without however limiting the scope thereof.

All of the catalysts prepared in examples 1 to 5 are prepared with the same content of element nickel. The support used for the preparation of each of these catalysts is a 6-alumina having a pore volume of 0.67 ml/g and a BET specific surface area equal to 140 m²/g.

### Example 1: Preparation of the Aqueous Solution of Ni Precursors

An aqueous solution of Ni precursors (solution S1) used for the preparation of catalysts A, B, C and D is prepared at 25° C. by dissolving 276 g of nickel nitrate Ni(NO₃)₂.6H₂O (supplied by Strem Chemicals®) in a volume of 100 ml of demineralized water. The solution S1, the NiO concentration of which is 19.0% by weight (relative to the weight of the solution), is obtained.

### Example 2 (Comparative): Preparation of a Catalyst a by Impregnation of Nickel Nitrate without Additives

The solution S1 prepared in example 1 is dry-impregnated (7.4 ml of solution) on 10 g of said alumina support. The solid thus obtained is subsequently dried in an oven at 120° C. for 16 hours and then calcined under a stream of air of 1 l/h/g of catalyst at 450° C. for 2 hours.

The calcined catalyst A thus prepared contains 13.8% by weight of the element nickel supported on alumina and it has nickel oxide crystallites, the mean diameter of which (determined by X-ray diffraction from the width of the diffraction line located at the angle 2θ=43°) is 15.2 nm.

### Example 3 (Comparative): Preparation of a Catalyst B by Successive Impregnation of Nickel Nitrate and then of 4-Oxopentanoic Acid (Levulinic Acid)

Catalyst B is prepared by impregnation of Ni nitrate (7.4 ml of solution) on said alumina support and then by impregnation of levulinic acid using a {levulinic acid/nickel} molar ratio equal to 0.4.

In order to do this, the solution S1 prepared in example 1 is dry-impregnated on said alumina support. The solid B1 thus obtained is then dried in an oven at 120° C. for 16 hours. An aqueous solution B′ is then prepared by dissolving 3.26 g of levulinic acid (CAS 123-76-2, supplied by Merck®) in 20 ml of demineralized water. This solution B′ is then dry-impregnated on 10 g of the previously prepared solid B1. The solid thus obtained is subsequently dried in an oven at 120° C. for 16 hours and then calcined under a stream of air of 1 l/h/g of catalyst at 450° C. for 2 hours.

The calcined catalyst B thus prepared contains 13.8% by weight of the element nickel supported on alumina and it has nickel oxide crystallites, the mean diameter of which is 5.2 nm.

### Example 4 (Invention): Preparation of a Catalyst C by Successive Impregnation of Nickel Nitrate and then of Levulinic Acid (4-Oxopentanoic Acid), with an Additive-to-Nickel Molar Ratio of 0.4, in the Gas Phase Using a Carrier Solid (According to Variant 2)

Catalyst C is prepared by impregnation of Ni nitrate on said alumina support and then by impregnation of levulinic acid in the gas phase using a {levulinic acid/nickel} molar ratio equal to 0.4. This method of preparation uses a carrier solid.

In order to do this, the solution S1 prepared in example 1 is dry-impregnated on said alumina support. The solid C1 thus obtained is then dried in an oven at 120° C. for 16 hours.

An aqueous solution C′ is then prepared by dissolving 3.26 g of levulinic acid (CAS 123-76-2, supplied by Merck®) in 20 ml of demineralized water. The solid C2 is obtained by dry impregnation of 7.4 ml of this solution C′ on said alumina support.

The solid C2 is then placed in a tubular reactor, for example a DN 50 mm quartz tube fitted with a frit, on a thin layer (approximately 1 cm). A bed of inert materials with a low surface area is then deposited (on a layer of a few cm, in this case SiC from the company AGP), followed by the second solid C1. A circulation of carrier gas (dry air in this case) is then carried out from the bottom to the top of the reactor (passing through C2 then through C1). A flow rate of 1 I/h/g is used; the temperature is increased to 120° C. over the zone containing the solid C2 and to 30° C. over that containing the solid C1. A vacuum is pulled in the system via a vane pump placed on the top of the quartz tube. The device is maintained for 8 hours with a vacuum of at least 50 mbar. The conditions are chosen to transfer the levulinic acid in vapor form from the solid C2 to the solid C1. At the end of the time necessary for the transfer, the solid C1 which has taken up the levulinic acid becomes the solid C.

The solid C thus obtained is then calcined under a stream of air of 1 l/h/g of catalyst at 450° C. for 2 hours.

The calcined catalyst C thus prepared contains 13.8% by weight of the element nickel supported on alumina and it has nickel oxide crystallites, the mean diameter of which is 4.9 nm.

### Example 5 (Invention): Preparation of a Catalyst D by Successive Impregnation of Nickel Nitrate and then of Levulinic Acid (4-Oxopentanoic Acid), with an Additive-to-Nickel Molar Ratio of 0.4, in the Gas Phase (According to Variant 1)

Catalyst D is prepared by impregnation of Ni nitrate on said alumina support and then by impregnation of levulinic acid in the gas phase using a {levulinic acid/nickel} molar ratio equal to 0.4.

In order to do this, the solution S1 prepared in example 1 is dry-impregnated on said alumina support. The solid D1 thus obtained is then dried in an oven at 120° C. for 16 hours.

Then, 3.26 g of levulinic acid (CAS 123-76-2, supplied by Merck®) are deposited pure and undiluted at the bottom of a saturator. Said saturator is connected to a quartz reactor wherein the solid D1 is placed on a porous frit in a monolayer of solid. The reactor is 5.5 cm in diameter for the 10 g of solid to be treated. The saturator/reactor assembly is brought to a uniform temperature. Under a stream of nitrogen (150 nl/h) which is injected at the base of the saturator, the temperature of the saturator/reactor assembly is adjusted to a temperature of 120° C. The temperature conditions are chosen so that the additive has a vapor pressure of at least 400 Pa. The whole thing is left under a stream of nitrogen at temperature for 8 hours. The system is then inerted, the saturator is bypassed, and air is then injected into the same assembly. The temperature of the reactor alone is increased (1° C./minute) under a stream of a 50/50 air/nitrogen mixture at 450° C. for 2 hours.

The calcined catalyst D thus prepared contains 13.8% by weight of the element nickel supported on alumina and it has nickel oxide crystallites, the mean diameter of which is 4.9 nm.

### Example 6: Evaluation of the Catalytic Properties of Catalysts a to D in Toluene Hydrogenation

Catalysts A to D described in the examples above are tested with respect to the toluene hydrogenation reaction.

The selective hydrogenation reaction is carried out in a 500 ml stainless steel autoclave which is provided with a magnetically-driven mechanical stirrer and which is able to operate under a maximum pressure of 100 bar (10 MPa) and temperatures of between 5° C. and 200° C.

Prior to its introduction into the autoclave, an amount of 2 ml of catalyst is reduced ex situ under a stream of hydrogen of 1 l/h/g of catalyst at 400° C. for 16 hours (temperature rise gradient of 1° C./min) and then it is transferred into the autoclave, with the exclusion of air. After addition of 216 ml of n-heptane (supplied by VWR®, purity >99% Chromanorm HPLC), the autoclave is closed, purged, then pressurized under 35 bar (3.5 MPa) of hydrogen and brought to the temperature of the test, which is equal to 80° C. At the time t=0, approximately 26 g of toluene (supplied by SDS®, purity >99.8%) are introduced into the autoclave (the initial composition of the reaction mixture is then toluene 6% by weight/n-heptane 94% by weight) and stirring is started at 1600 rev/min. The pressure is kept constant at 35 bar (3.5 MPa) in the autoclave using a storage cylinder located upstream of the reactor.

The progress of the reaction is monitored by taking samples from the reaction medium at regular time intervals: the toluene is completely hydrogenated to give methylcyclohexane. The hydrogen consumption is also monitored over time by the decrease in pressure in a storage cylinder located upstream of the reactor. The catalytic activity is expressed in moles of H₂ consumed per minute and per gram of Ni.

The catalytic activities measured for catalysts A to D are reported in Table 1 below. They are related back to the catalytic activity measured for catalyst A (A_{HYD1}).

TABLE 1

| | | Additive | Mean size of the | |
|---|---|---|---|---|
| | Additive | introduction | NiO crystallites | A_{HYD1} |
| Catalyst | used | method | (nm) | (%) |
| A (not in | — | — | 15.2 | 100 |
| accordance | | | | |
| with the | | | | |
| invention) | | | | |
| B (not in | Levulinic | Post liquid | 5.2 | 260 |
| accordance | acid | impregnation | | |
| with the | | | | |
| invention) | | | | |
| C (in | Levulinic | Post gas | 4.9 | 305 |
| accordance | acid | impregnation, | | |
| with the | | via carrier | | |
| invention) | | solid | | |
| D (in | Levulinic | Post gas | 4.9 | 300 |
| accordance | acid | impregnation | | |
| with the | | | | |
| invention) | | | | |

The results shown in Table 1 demonstrate that catalysts B, C and D, prepared in the presence of an organic compound (having at least one carboxylic acid type function), are more active than catalyst A prepared in the absence of this type of organic compound. This effect is related to the decrease in the size of the Ni particles. The additive introduction method also has an effect, which is not related to the size of the Ni particles, on the activity of the catalyst. A reduction in the nickel aluminate content is observed following this additive introduction method in the gas phase.

### Example 7: Evaluation of the Catalytic Properties of Catalysts a to D in the Selective Hydrogenation of a Mixture Containing Styrene and Isoprene

Catalysts A to D described in the examples above are tested with regard to the reaction for the selective hydrogenation of a mixture containing styrene and isoprene.

The composition of the feedstock to be selectively hydrogenated is as follows: 8% by weight of styrene (supplied by Sigma Aldrich®, purity 99%), 8% by weight of isoprene (supplied by Sigma Aldrich®, purity 99%) and 84% by weight of n-heptane (solvent) (supplied by VWR®, purity >99% Chromanorm HPLC). This feedstock also contains sulfur-containing compounds in a very small content: 10 ppm by weight of sulfur introduced in the form of pentanethiol (supplied by Fluka®, purity >97%) and 100 ppm by weight of sulfur introduced in the form of thiophene (supplied by Merck®, purity 99%). This composition corresponds to the initial composition of the reaction mixture. This mixture of model molecules is representative of a pyrolysis gasoline.

The selective hydrogenation reaction is carried out in a 500 ml stainless steel autoclave which is provided with a magnetically-driven mechanical stirrer and which is able to operate under a maximum pressure of 100 bar (10 MPa) and temperatures of between 5° C. and 200° C.

Prior to its introduction into the autoclave, an amount of 3 ml of catalyst is reduced ex situ under a stream of hydrogen of 1 l/h/g of catalyst at 400° C. for 16 hours (temperature rise gradient of 1° C./min) and then it is transferred into the autoclave, with the exclusion of air. After addition of 214 ml of n-heptane (supplied by VWR®, purity >99% Chromanorm HPLC), the autoclave is closed, purged, then pressurized under 35 bar (3.5 MPa) of hydrogen and brought to the temperature of the test, which is equal to 30° C. At the time t=0, approximately 30 g of a mixture containing styrene, isoprene, n-heptane, pentanethiol and thiophene are introduced into the autoclave. The reaction mixture then has the composition described above and stirring is started at 1600 rev/min. The pressure is kept constant at 35 bar (3.5 MPa) in the autoclave using a storage cylinder located upstream of the reactor.

The progress of the reaction is monitored by taking samples from the reaction medium at regular time intervals: the styrene is hydrogenated to give ethylbenzene, without hydrogenation of the aromatic ring, and the isoprene is hydrogenated to give methylbutenes. If the reaction is prolonged for longer than necessary, the methylbutenes are in their turn hydrogenated to give isopentane. The hydrogen consumption is also monitored over time by the decrease in pressure in a storage cylinder located upstream of the reactor. The catalytic activity is expressed in moles of H₂ consumed per minute and per gram of Ni.

The catalytic activities measured for catalysts A to D are reported in Table 2 below. They are related back to the catalytic activity measured for catalyst A (A_{HYD2}).

TABLE 2

| | | Additive | Mean size of the | |
|---|---|---|---|---|
| | Additive | introduction | NiO crystallites | A_{HYD2} |
| Catalyst | used | method | (nm) | (%) |
| A (not in | — | — | 15.2 | 100 |
| accordance | | | | |
| with the | | | | |
| invention) | | | | |
| B (not in | Levulinic | Post liquid | 5.2 | 240 |
| accordance | acid | impregnation | | |
| with the | | | | |
| invention) | | | | |
| C (in | Levulinic | Post gas | 4.9 | 270 |
| accordance | acid | impregnation | | |
| with the | | via carrier | | |
| invention) | | solid | | |
| D (in | Levulinic | Post gas | 4.9 | 285 |
| accordance | acid | impregnation | | |
| with the | | | | |
| invention) | | | | |

The results shown in Table 2 demonstrate that catalysts B, C and D, prepared in the presence of an organic compound (having at least one carboxylic acid type function), are more active than catalyst A prepared in the absence of this type of organic compound. This effect is related to the decrease in the size of the Ni particles. The additive introduction method also has an effect, which is not related to the size of the Ni particles, on the activity of the catalyst. A reduction in the nickel aluminate content is observed following this additive introduction method in the gas phase.

1. A process for the hydrogenation of at least one polyunsaturated compound containing at least 2 carbon atoms per molecule, such as diolefins and/or acetylenics and/or aromatic or polyaromatic compounds, contained in a hydrocarbon feedstock having a final boiling point of less than or equal to 650° C., which process being carried out at a temperature of between 0 and 350° C., at a pressure of between 0.1 and 20 MPa, at a hydrogen/(compound to be hydrogenated) molar ratio between 0.1 and 1000 and at an hourly space velocity HSV of between 0.05 and 40 000 h⁻¹ in the presence of a catalyst comprising a porous support and an active phase comprising at least one group VIII metal, said active phase not comprising a group VIB metal, said catalyst being prepared according to at least the following steps:
a) at least one organic compound containing oxygen and/or nitrogen, but not comprising sulfur, is added to the porous support;
b) a step of bringing said porous support into contact with at least one solution containing at least one salt of a precursor of the phase comprising at least one group VIII metal is carried out;
c) the porous support obtained at the end of step b) is dried;
characterized in that step a) is carried out before or after steps b) and c) and is carried out by bringing together said porous support and said organic compound under conditions of temperature, pressure and duration such that a fraction of said organic compound is transferred in the gaseous state to the porous support.

2. The process as claimed in claim 1, wherein step a) is carried out by simultaneously bringing together said porous support and said organic compound in the liquid state and without physical contact, at a temperature below the boiling point of said organic compound and under conditions of pressure and duration such that a fraction of said organic compound is transferred in the gaseous state to the porous support.

3. The process as claimed in claim 2, wherein step a) is carried out by means of a unit for adding said organic compound comprising a first compartment and a second compartment that are in communication so as to allow the passage of a gaseous fluid between the compartments, the first compartment containing the porous support and the second compartment containing the organic compound in the liquid state.

4. The process as claimed in claim 3, wherein the unit comprises a chamber that includes the first and second compartments, the two compartments being in gaseous communication.

5. The process as claimed in claim 3, wherein the unit comprises two chambers that respectively form the first and second compartments, the two chambers being in gaseous communication.

6. The process as claimed in claim 3, wherein step a) is carried out in the presence of a stream of a carrier gas circulating from the second compartment into the first compartment.

7. The process as claimed in claim 1, wherein step a) is carried out by bringing together said porous support with a porous solid comprising said organic compound under conditions of temperature, pressure and duration such that a fraction of said organic compound is transferred gaseously from said porous solid to said porous support.

8. The process as claimed in claim 7, wherein step a) is carried out by bringing together said porous support with said porous solid comprising said organic compound, without physical contact.

9. The process as claimed in claim 7, wherein, in step a), the porous support and the porous solid comprising said organic compound are of different porosity and/or of different chemical nature.

10. The process as claimed in claim 7, wherein, at the end of step a), the porous solid containing the organic compound is separated from said porous support and is returned to step a).

11. The process as claimed in claim 1, wherein said organic compound is chosen from compounds comprising one or more chemical functions chosen from a carboxylic acid, alcohol, ester, aldehyde, ketone, ether, carbonate, amine, azo, nitrile, imine, amide, carbamate, carbamide, amino acid, ether, dilactone or carboxyanhydride function.

12. The process as claimed in claim 11, wherein said organic compound comprises at least one carboxylic function chosen from formic acid, ethanedioic acid (oxalic acid), propanedioic acid (malonic acid), pentanedioic acid (glutaric acid), hydroxyacetic acid (glycolic acid), 2-hydroxypropanoic acid (lactic acid), 2-hydroxypropanedioic acid (tartronic acid), 2-hydroxybutanedioic acid (malic acid), 2-hydroxypropane-1,2,3-tricarboxylic acid (citric acid), 2,3-dihydroxybutanedioic acid (tartaric acid), 2,2′-oxydiacetic acid (diglycolic acid), 2-oxopropanoic acid (pyruvic acid) and 4-oxopentanoic acid (levulinic acid).

13. The process as claimed in claim 11, wherein said organic compound comprises at least one alcohol function chosen from methanol, ethanol, phenol, ethylene glycol, propane-1,3-diol, butane-1,4-diol, pentane-1,5-diol, hexane-1,6-diol, glycerol, xylitol, mannitol, sorbitol, pyrocatechol, resorcinol, hydroquinol, diethylene glycol, triethylene glycol, polyethylene glycols having an average molar mass of less than 600 g/mol, glucose, mannose, fructose, sucrose, maltose and lactose, in any of their isomeric forms.

14. The process as claimed in claim 11, wherein said organic compound comprises at least one ester function chosen from a γ-lactone or a δ-lactone containing between 4 and 8 carbon atoms, the γ-butyrolactone, γ-valerolactone, δ-valerolactone, γ-caprolactone, 6-caprolactone, γ-heptalactone, δ-heptalactone, γ-octalactone, δ-octalactone, methyl methanoate, methyl acetate, methyl propanoate, methyl butanoate, methyl pentanoate, methyl hexanoate, methyl octanoate, methyl decanoate, methyl laurate, methyl dodecanoate, ethyl acetate, ethyl propanoate, ethyl butanoate, ethyl pentanoate, ethyl hexanoate, dimethyl oxalate, dimethyl malonate, dimethyl succinate, dimethyl glutarate, dimethyl adipate, diethyl oxalate, diethyl malonate, diethyl succinate, diethyl glutarate, diethyl adipate, dimethyl methylsuccinate, dimethyl 3-methylglutarate, methyl glycolate, ethyl glycolate, butyl glycolate, benzyl glycolate, methyl lactate, ethyl lactate, butyl lactate, tert-butyl lactate, ethyl 3-hydroxybutyrate, ethyl mandelate, dimethyl malate, diethyl malate, diisopropyl malate, dimethyl tartrate, diethyl tartrate, diisopropyl tartrate, trimethyl citrate, triethyl citrate, ethylene carbonate, propylene carbonate, trimethylene carbonate, diethyl carbonate, diphenyl carbonate, dimethyl dicarbonate, diethyl dicarbonate and di-tert-butyl dicarbonate, in any of their isomeric forms.

15. The process as claimed in claim 11, wherein said organic compound comprises at least one amine function chosen from ethylenediamine, diaminohexane, tetramethylenediamine, hexamethylenediamine, tetramethylethylenediamine, tetraethylethylenediamine, diethylenetriamine and triethylenetetramine.

16. The process as claimed in claim 11, wherein said organic compound comprises at least one amide function chosen from formamide, N-methylformamide, N,N-dimethylformamide, N-ethylformamide, N,N-diethylformamide, acetamide, N-methylacetamide, N,N-dimethylmethanamide, N,N-diethylacetamide, N,N-dimethylpropionamide, propanamide, 2-pyrrolidone, N-methyl-2-pyrrolidone, γ-lactam, caprolactam, acetylleucine, N-acetylaspartic acid, aminohippuric acid, N-acetylglutamic acid, 4-acetamidobenzoic acid, lactamide and glycolamide, urea, N-methylurea, N,N′-dimethylurea, 1,1-dimethylurea, and tetramethylurea, according to any one of their isomeric forms.

17. The process as claimed in claim 11, wherein said organic compound comprises at least one carboxyanhydride function chosen from the group of the O-carboxyanhydrides consisting of 5-methyl-1,3-dioxolane-2,4-dione and 2,5-dioxo-1,3-dioxolane-4-propanoic acid, or from the group of the N-carboxyanhydrides consisting of 2,5-oxazolidinedione and 3,4-dimethyl-2,5-oxazolidinedione.

18. The process as claimed in claim 11, wherein said organic compound comprises at least one dilactone function chosen from the group of the cyclic dilactones having 4 ring members consisting of 1,2-dioxetanedione, or from the group of the cyclic dilactones having 5 ring members consisting of 1,3-dioxolane-4,5-dione, 1,5-dioxolane-2,4-dione, and 2,2-dibutyl-1,5-dioxolane-2,4-dione, or from the group of the cyclic dilactones having 6 ring members consisting of 1,3-dioxane-4,6-dione, 2,2-dimethyl-1,3-dioxane-4,6-dione, 2,2,5-trimethyl-1,3-dioxane-4,6-dione, 1,4-dioxane-2,5-dione, 3,6-dimethyl-1,4-dioxane-2,5-dione, 3,6-diisopropyl-1,4-dioxane-2,5-dione, and 3,3-ditoluyl-6,6-diphenyl-1,4-dioxane-2,5-dione, or from the group of the cyclic dilactones having 7 ring members consisting of 1,2-dioxepane-3,7-dione, 1,4-dioxepane-5,7-dione, 1,3-dioxepane-4,7-dione and 5-hydroxy-2,2-dimethyl-1,3-dioxepane-4,7-dione.

19. The process as claimed in claim 11, wherein said organic compound comprises at least one ether function chosen from the group of linear ethers consisting of diethyl ether, dipropyl ether, dibutyl ether, methyl tert-butyl ether, diisopropyl ether, di-tert-butyl ether, methoxybenzene, phenyl vinyl ether, isopropyl vinyl ether and isobutyl vinyl ether, or from the group of cyclic ethers consisting of tetrahydrofuran, 1,4-dioxane and morpholine.

20. The process as claimed in claim 1, said process being a process for the hydrogenation of at least one aromatic or polyaromatic compound contained in a hydrocarbon feedstock having a final boiling point of less than or equal to 650° C., said process being carried out in the gas phase or in the liquid phase, at a temperature of between 30 and 350° C., at a pressure of between 0.1 and 20 MPa, at a hydrogen/(aromatic compounds to be hydrogenated) molar ratio between 0.1 and 10 and at an hourly space velocity HSV of between 0.05 and 50 h⁻¹.

21. The process as claimed in claim 1, wherein said process is a process for the selective hydrogenation of polyunsaturated compounds contained in a hydrocarbon feedstock having a final boiling point of less than or equal to 300° C., which process being carried out at a temperature of between 0 and 300° C., at a pressure of between 0.1 and 10 MPa, at a hydrogen/(polyunsaturated compounds to be hydrogenated) molar ratio of between 0.1 and 10 and at an hourly space velocity of between 0.1 and 200 h⁻¹ when the process is carried out in the liquid phase, or at a hydrogen/(polyunsaturated compounds to be hydrogenated) molar ratio of between 0.5 and 1000 and at an hourly space velocity of between 100 and 40 000 h⁻¹ when the process is carried out in the gas phase.
